(19) United States
(12) Patent Application Publication
(10) Pub. No.: US 2015/0257683 A1
(43) Pub. Date: Sep. 17, 2015

(54) APPARATUS FOR TESTING HEARING

(71) Applicant: Audiology-Online Ltd, Somerset (GB)

(72) Inventors: Mark Ashmore, Ashcott (GB); Ray Weinstein, Devon (GB)

(73) Assignee: Audiology-Online Ltd, Somerset (GB)

(21) Appl. No.: 14/208,178

(22) Filed: Mar. 13, 2014

(51) Int. Cl. A61B 5/12; A61B 7/04; A61B 5/00

(52) U.S. Cl. CPC A61B 5/123; A61B 5/0022; A61B 5/7475; A61B 5/7282; A61B 5/7405; A61B 5/7228; A61B 5/6803; A61B 7/04

(57) ABSTRACT

Apparatus for testing hearing is arranged to deliver audio tones over the Internet to be output at a receiving station such as a user's computer. The apparatus firstly constructs audio tones for transmission out of sets of components recorded at different modulation levels for each frequency. This allows significantly finer control over the sound level of the audio tones at the receiving station than would normally be available at the receiving end. Secondly, the apparatus supports a feedback loop, based on a calibrated audio tone delivered for output at the receiving station and transmitted as audible sound back to the hearing test apparatus via a voice activated modem, the apparatus sending display data to the receiving station representing the sound level of that audible sound such that a user can adjust one or more parameters of the receiving station so as to bring the sound level of the audible sound, indicated by the display data, to a calibrated level. Alternatively, the apparatus might adjust the parameters of the receiving station directly, using a Java applet or the like. Calibration can additionally or instead be provided by means of an audio pickup for measuring and feeding back the level of audio output at audio delivery equipment in use to deliver audio tones to a user.

This invention relates to apparatus for testing hearing.

Hearing tests have been known and used for some time. A form of apparatus for use in testing hearing is known as an audiometer. An audiometer generates a pure tone signal. In a known method of testing hearing in a subject, an audiometer is used to present the pure tone signal to the subject in a range of frequencies and at various intensities. The subject indicates, for instance by pressing a button that a presented tone signal is heard and the lowest intensities at which the tones are each heard are recorded. These lowest intensities are plotted on a scale to produce an audiogram for the subject, showing the threshold of hearing at each frequency where the presented tone signal is just perceived by the subject.

It is also known to test hearing over the Internet by sending a tone signal to an online user. The user responds via their browser. These arrangements are generally more convenient to the user than the traditional form of test in which an operator, specialised equipment and the user have to be in the same location at the same time. They are also ad hoc however because the direct link between tone generation and the user is lost and the tones generated by the available multitude of combinations of headphones and soundcards may not match the calibrated pure tone signals delivered by a calibrated audiometer.

According to a first aspect of embodiments of the present invention, there is provided hearing test apparatus for transmitting audio tones over a network for playing as audible sound at a receiving station and for receiving inputs over the network in relation to the audible sound, the apparatus comprising:

i) an audio tone store for storing recorded audio tones,
ii) a transmitter for transmitting selected tones via the network to the receiving station,
iii) a receiver for receiving inputs from the receiving station via the network in relation to transmitted audio tones, and
iv) a processor for processing and responding to received inputs so as to carry out a hearing test and generate hearing test results,
wherein the audio tone store is structured to store each audio tone as a set of two or more audio tone components having the same frequency, each component having a different modulation level, and the apparatus further comprises:
iii) an audio tone selector for selecting in accordance with received inputs, for each transmitted audio tone, one or more audio tone components from one of said sets for transmission via the network.

The network may comprise a public network such as the Internet, giving access to a considerable number of potential receiving stations.

The accuracy of the term “same frequency” in this context will be determined by the required accuracy of the results and/or by the means used to generate the audio tone components. If an audiometer is used, then the frequencies are likely to be accurately the same but other sources might generate frequencies that are not pure or exactly matched in practice.

Embodiments of the invention in its first aspect allow audio tones to be transmitted via the Internet in a manner that can be tailored to the user's audio environment at the receiving station so as to ensure that the user's receiving station, together with any ancillary equipment such as headphones, will deliver sounds to the user's ear which match the sound pressure levels required to correspond with a calibrated audiometer.

An audio tone store might be structured to store each set of audio tone components as a set of two, three, four or more sound files at the same frequency, for example each sound file of a set having been recorded at different modulation levels. These sets of sound files for each frequency can be used with varying playback volumes and each transmitted audio tone may comprise one or more components selected from a set recorded at different modulation levels. This gives a much finer control over the output level of the audio tones at the receiving station (usually a user's computer) than would be available using just the volume control on the user's computer itself and this fine control can also be used to adapt the audio tones to the user's specific audio environment, for example, giving a desired level of reproducibility and standardisation.

Typically, a receiving station might comprise a home or office computer with a standard sound card and an ordinary earpiece or headphones. Alternatively the receiving station may comprise a home or office computer with a known set of headphones which incorporate a pair of microphones and a sound card. Embodiments of the invention support an online or Internet-accessible hearing evaluation tool useable by anyone with a basic setup and an Internet connection. The accuracy of the results might not necessarily be appropriate for clinical use but can be comparable to results achieved using a known audiometer locally, that is at the user location, for screening and/or testing.

There are various factors that mean the standard output of known audiometers cannot simply be recorded and transmitted over the Internet. As mentioned above, volume control can be a problem. For example, the conventional volume control on a standard computer is typically only software-adjustable in 1 step increments. If an audiometric tone signal has been recorded at 100% modulation (0 dB electronic output level), 1% of 0 dB can be far too great as a minimum unit for use in a hearing test. In embodiments of the invention, instead of using the conventional volume control, it is possible to provide a volume control command input via the computer at the receiving station to send volume control commands to the transmission apparatus to set the playback volumes of the sets of sound files. This volume control command input can be designed to exploit the much finer control over the output level offered by the sets of sound files.

In embodiments of the invention in its first aspect therefore, the transmitting apparatus preferably has an input for receiving volume control data from the receiving station, in use of the apparatus, which data is processed by the audio tone selector for use in selecting the audio tone components and/or output levels to be transmitted via the Internet.

Other problems in delivering a reproducible output level include variances in computer soundcards, the wide choice of headphone or ear insert type and headphone component manufacture. In embodiments of the invention, one or more of all these variables can be accounted for in order to give a degree of reproducibility and standardised results to the individual.

The use of a set of audio tone components for each frequency, according to an embodiment of the invention, allows these other factors to be taken into account. For example, by setting headphone type, or by calibrating the user's system at the receiving station at the outset, a suitable mix of audio tone components and/or output levels can be selected for transmission at each frequency.

In order to deal with variability of the user's audio environment at the receiving station, embodiments of the invention benefit from including a reference data store, accessible to or stored at the transmission apparatus, for use by the audio tone selector in selecting the audio tone components and/or output levels to be transmitted via the Internet. The user at the receiving station might be provided for example with an on-screen headphone selector such as a drop down menu. By changing the on-screen system headphone selection, for instance, the user causes commands to be sent from their computer back to the transmitting apparatus and the transmitting apparatus responds by selecting the mix of audio tone components and/or output levels. This allows pre-calibration to be done with reference to such factors as the use of different headphones. Thus the input for receiving volume control data might also be adapted to receive calibration data, again for use by the audio tone selector in selecting the audio tone components and/or output levels.

It has been found that a suitable set of audio tone components for hearing test purposes, being sufficient to provide a useful range of output level, can be provided with one of them recorded at high modulation, perhaps 90% to 100%, and the rest of them recorded at less than 10% modulation. Indeed, a set of components in which at least one component lies in each of the ranges 1.0-10%, 0.1-1.0% and less than 0.1% modulation has been found to provide a useful range. Further, it has been found preferable to use at least four components in order to have sufficient flexibility to provide a good match across a range of different audio environments at the receiving station.

In order to support potentially very accurate calibration with regard to the user's equipment at the receiving station, embodiments of the invention in a second aspect might comprise hearing test apparatus for transmitting audio tones over a network for playing as audible sound at a receiving station and for receiving inputs over the network in relation to the audible sound, further comprising calibration apparatus for use in calibrating the volume of audible output of the receiving station in response to one or more calibration audio tones transmitted by the transmitter, the calibration apparatus comprising:

a) an audio tone input for receiving a copy of the audible output from the receiving station; and
b) a volume data generator for generating volume data from the received copy, the calibration apparatus being arranged to provide, in use, a feedback loop in which a copy of the audible output of the receiving station, in response to one or more calibration audio tones transmitted by the transmitter, is returned to the audio tone transmission apparatus, and used to generate volume data for use in turn at the receiving station in adjusting one or more sound output levels of the receiving station so as to calibrate the volume of the audible output of the receiving station.

The volume data might be used for instance to generate a volume display output for transmission to the receiving station, for use by means of user inputs in said adjustment of one or more sound output levels of the receiving station.

The copy of the audible output might be transmitted from the receiving station to the transmission apparatus by means of a telephone connection, using the microphone of a standard telephone to pick up the audible output of the user's equipment and send a copy back to the transmission apparatus. The volume display output at the transmission apparatus might then comprise a sound level meter for example, for use in generating volume data to support the volume display output.

The calibration apparatus however preferably comprises a voice activated modem (“VAM”). The telephone of the user can then be used to connect to the VAM to send the copy of the audible output back to the transmission apparatus. The VAM provides analogue to digital conversion of the copy which can be digitally analysed by the volume data generator.

In a variation, the calibration apparatus might comprise the audio tone input and volume data generator described above, but replace the volume display output with a volume control output for transmitting volume control data or commands to the receiving station in accordance with the volume data for use in direct adjustment of one or more sound output levels of the receiving station so as to calibrate the volume of the audible output of the receiving station. For example, the volume of audio outputs at a receiving station that comprises a computer is often controlled by the output level of a sound card. It is possible, for example by installing local software, for the volume control data or commands sent from the hearing test apparatus to have direct control over sound card levels at the receiving station via such local software.

Another variable which can introduce inaccuracy into a hearing test arises because the audio delivery equipment for delivering audio tones to the user, such as headphones or insert ear phones, does not do that in a fully predictable manner Embodiments of the invention in a third aspect, for dealing with this inaccuracy, might comprise hearing test apparatus comprising:

i) a transmitter for transmitting selected audio tones having different respective volumes via a network to a receiving station,
ii) audio delivery equipment for use at the receiving station in delivering audio tones to the user's ear,
iii) a receiver for receiving inputs from the receiving station via the network in relation to transmitted audio tones, and
v) a processor for processing and responding to received inputs so as to carry out a hearing test,
the hearing test apparatus further comprising calibration apparatus for use in calibrating the volume of delivered audible output to a user's ear in response to audio tones transmitted by the transmitter, wherein the audio delivery equipment comprises an audio pickup for picking up transmitted audio tones at delivery to the user's ear, for use in providing feedback to the processor enabling volume calibration of the audio tones in relation to the audio delivery equipment in use.

The audio delivery equipment might comprise for example headphones or an insert ear phone. The audio pickup might comprise a microphone mounted in the audio delivery equipment for picking up audio tones at the point of delivery to the user's ear for use in providing a volume indication.

Embodiments of the invention can provide hearing test results with improved accuracy in a number of ways, including by means of remote adjustment of user equipment rather than relying on local volume control and by offering significant calibration improvements/features.

It is to be understood that any feature described in relation to any one aspect or to any one embodiment of the invention may be used alone, or in combination with other features described, in relation to the same or one or more other aspects or embodiments of the invention if appropriate.

Hearing test equipment according to an embodiment of the invention will now be described, by way of example only, with reference to the accompanying figures in which:

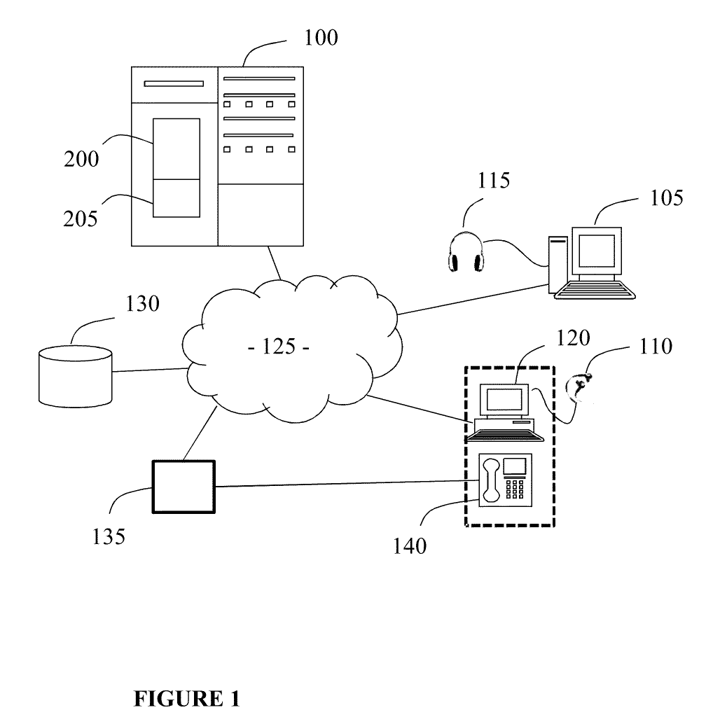

FIG. 1 shows a schematic block diagram of the hearing test equipment connected for use over the Internet;

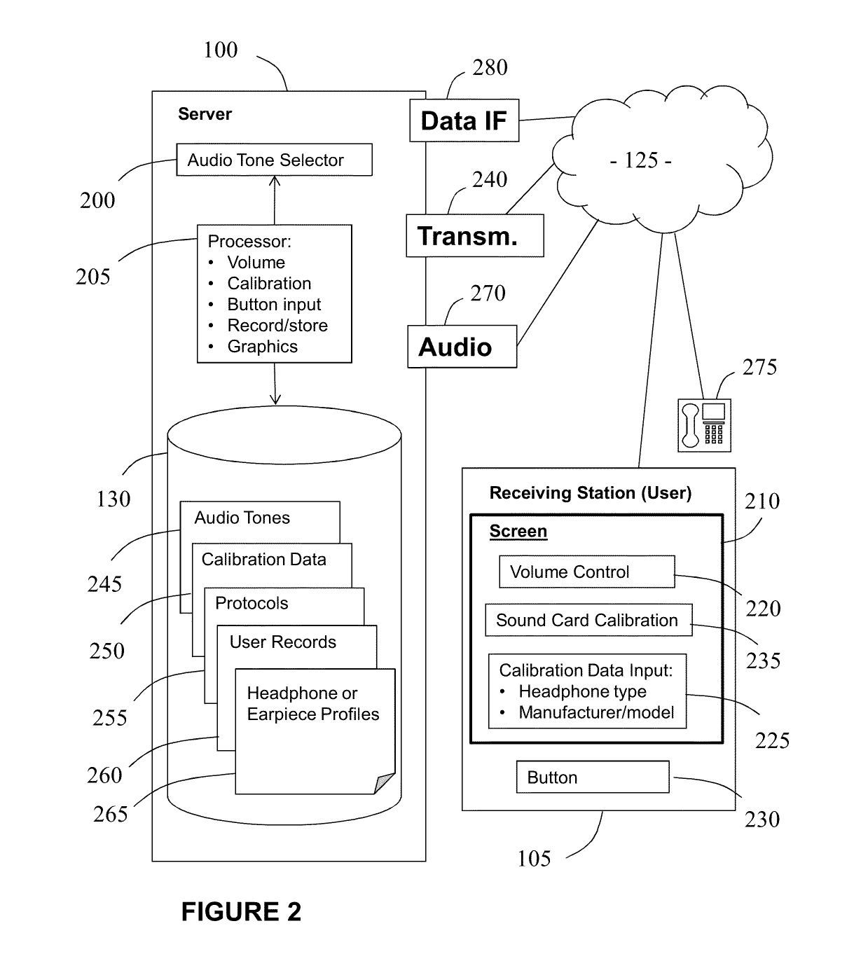

FIG. 2 shows a block diagram of internal components of the hearing test equipment, together with functions provided at a user's receiving station;

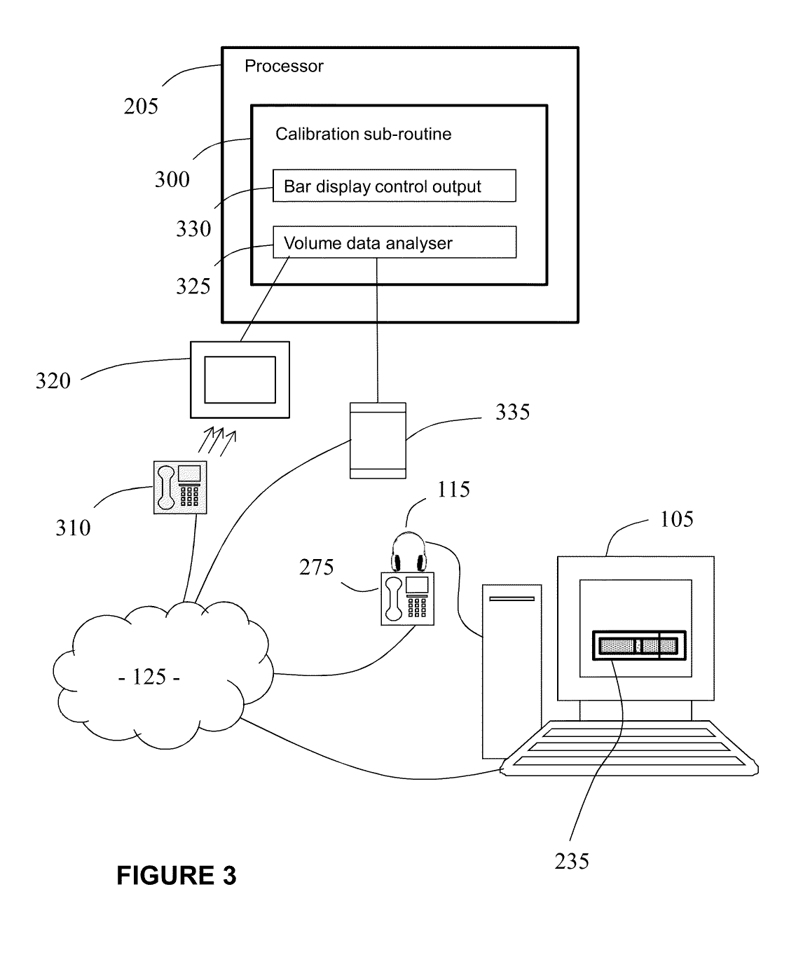

FIG. 3 shows a schematic block diagram of sound card calibration apparatus for use in the hearing test equipment;

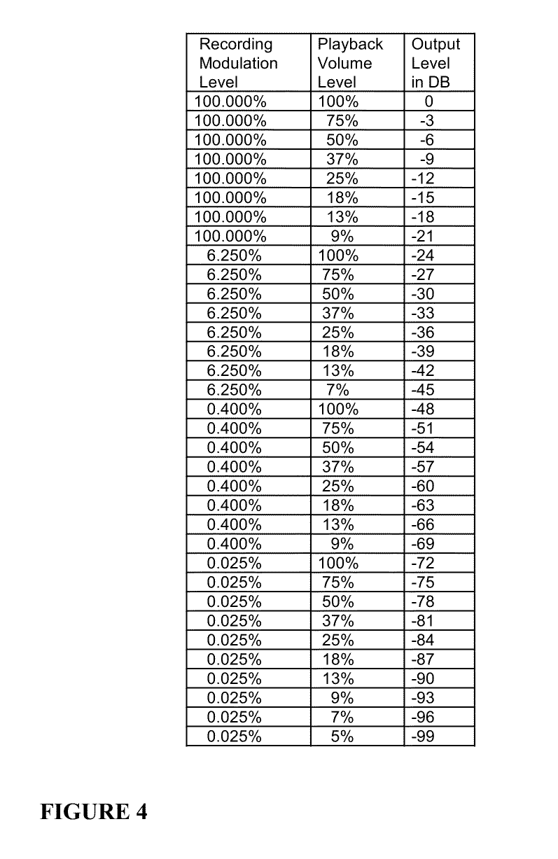

FIG. 4 shows a table of sets of audio tone components for use in the hearing test equipment; and

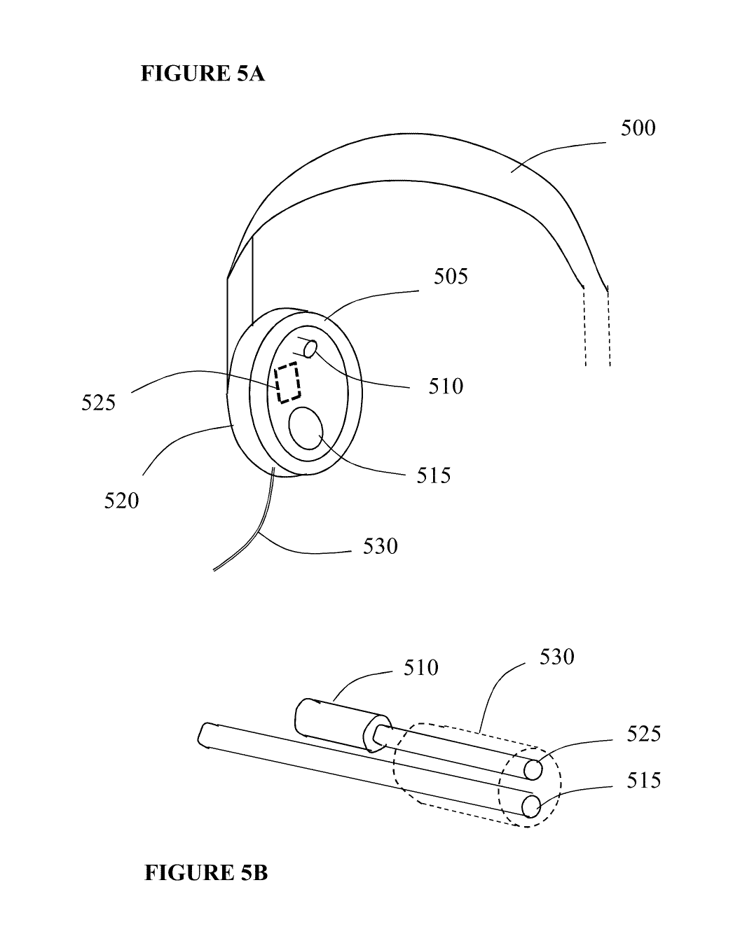

FIGS. 5A and 5B show schematically a headset and ear insert for calibrating user equipment in use of the hearing test equipment of FIG. 1.

Referring to FIG. 1, the hearing test equipment comprises software 200, 205 installed on a server 100 connected to the Internet 125. The equipment has access to a database 130, also via the Internet as shown, and user receiving stations 105, 120 of different types are connected for access over the Internet to the hearing test equipment. As shown, the user receiving stations 105, 120 are each equipped with different types of headphone, for example these being over the ear headphones 115 and insert headphones 110 respectively.

It will be understood that embodiments of the invention are not limited to use over the Internet and might indeed be connected over other networks.

In general, to carry out a hearing test, the user accesses a website and requests an online test, for example by clicking on a button. This runs one or more software-controlled processes on the server 100.

Referring to FIG. 2, the hearing test equipment established on the server 100 comprises a processor 205, an audio tone selector 200 and a data store 130. (As shown, these are all on the same server 100 but could be distributed to include other platforms.) The server 100 also has a data interface 280 for communicating over the Internet, a transmitter 240 for sending audio tones via the Internet 125 and an audio tone input 270, these being generally of known type. The processor 205 deals with interaction with a receiving station 105 such as a user's computer, in known manner, for instance by use of commands, forms, menus and graphical screen displays. The data interface 280 acts as a receiver for receiving inputs from the receiving station 105 in relation to sent audio tones or for other purposes related to a hearing test. The processor 205 also runs the audio test routines according to protocols 255 stored in the database 130, including calibration and records. The user can send commands and data to the processor 205, also in known manner, by use of their existing interface controls such as keyboard, mouse and audio input, to respond both to on-screen displays 220, 225 and audio transmissions transmitted by the server 100.

The audio tone selector 200 responds to the processor 205 to deliver audio tones for transmission to the receiving station 105 which have appropriate content and output level and this is further described below.

The data store 130 holds records of various types and in particular:
audio tones 245 for transmission over the Internet
calibration data 250 for interpreting user inputs identifying equipment types into audio tone requirements, including for example headphone profiles
protocols 255 for use by the processor 205 in running hearing tests
user records 260, such as name and contact details and hearing test results

The structuring of the stored audio tones 245 is important. As mentioned above, it is not possible to use a single audio tone at each frequency, recorded at one modulation level, and cover the full range of output levels with enough accuracy for a hearing test. Referring to FIG. 4, the audio tones 245 are therefore stored in sets of four components for each frequency. The components are recorded using different modulation levels, these being shown in the left-hand column as 100.000%, 6.250%, 0.400% and 0.025%. These values equate to sounds recorded at 0 dB modulation, −24 dB modulation, −48 dB modulation and −72 dB modulation. Each of these sound files can be played back at different playback volume levels, for instance eight or nine different levels, to give a finely graded variation in output level in dB as shown in the table in FIG. 4. Each audio tone 245 can therefore be described by the combination of its recording modulation level and playback volume, these being listed as “sounder levels” in Table 1 below.

Referring again to FIG. 2, the receiving station 105 will usually be equipped with whatever equipment the user has available, such as a computer, and will therefore have a screen 210 and a pre-installed network browser for accessing applications over the Internet. In embodiments of the invention, the user will access via the Internet 125 a hearing test application run by the processor 205 which will offer a volume control 220, sound card calibration meter 235 and other calibration data input 225 in a suitable manner for interactive display on the user's screen 210. Additionally, the hearing test application can receive via the data interface 280 commands via the user's keyboard, for example via an assigned key 230, to act as a button to press in response to hearing audio tones 245 delivered to the receiving station 105.

The playback volume control 220 might be displayed as a dial or slider that the user can move to higher and lower values prior to a hearing test (or might be automatically set during a calibration routine), thereby transmitting data to the processor 205 for use in controlling the audio tone selector 200 to send tones at different output levels to the user's computer 105.

Referring additionally to FIG. 3, the sound card calibration meter 235 might be displayed as a bar, coloured generally red with a green central portion, and a line or needle which can be moved along the bar under the control of a calibration sub-routine 300 available to the processor 205. Alternatively, the calibration meter 235 can be automated, thus not requiring a display on the user's screen 210 or interaction by the user.

The other calibration data input 225 will usually be a drop-down menu or the like, so that the user can select amongst known options to identify aspects of their equipment such as headphone type, brand and model.

Calibration techniques, including use of the calibration meter 235 and the calibration sub-routine 300 in calibrating the user's sound card and optionally their headphones or insert earphones, are further described below.

### Calibration by Reference: Headphones and Earpieces

Audiometers are calibrated to deliver a combination of known intensities and frequencies at a person's ear using a known and calibrated set of headphones and/or insert earpieces. To achieve the same intensity for headphones compared with insert earpieces, a tone of different intensity from the audiometer will be required to deliver the same sound at the person's ear. In simple terms a sound at a given level through a headphone placed over the ear would be heard more loudly if that same sound was instead delivered to the ear via an insert earpiece. This is because, with an insert earpiece, the same amount of energy is delivered to a smaller volume of air (since the insert earpiece seals the ear canal) and the sound is perceived as louder. There will also be variation between headphone or earpiece types and brands.

In embodiments of the invention, correct selection of the transmitted audio tones 245 to accommodate different audio environments at the receiving station is important, indeed can be critical, in achieving accurate results from the hearing tests, and the data store 130 holds reference data comprising calibration data records 250 for use by the processor 205 and audio tone selector 200 in order to match tone transmission to the user's audio environment. In a setup phase of use of the equipment, the user is asked to use the “other calibration data input” 225 to enter calibration data to indicate factors affecting their audio environment, such as headphone type and brand data. The calibration data can be interpreted by reference to a calibration data record 250 in the database 130. This can be used by the audio tone selector 200 in selecting sound files and playback volume levels from the table of FIG. 4.

It is possible to develop a library of audio tones 245 for storage in the database 130 which can be matched to different headphones and earpieces as follows. The level of sound produced at the ear by an audiometer is measured and matched to that produced using the hearing test equipment 200, 205, using a known type of calibrated real ear measurement (“REM”) machine, such as a Unity 2 REM system, together with a laptop. The routine is as follows:
1) Measure and record the sound at an ear of a sound from the audiometer using the REM machine
2) Match that sound (and record) at the ear by use of the audio tone selector 200 of the hearing test equipment to transmit sound via the Internet 125 to a set of headphones for which calibration data is required.
3) Repeat for all required frequencies and levels (eg) 20 dB-80 dB in 5 dB steps at 500 Hz, 1000 Hz, 2000 Hz and 4000 Hz and others as required.
4) Repeat for other headphone systems
5) Repeat using different ears

Data can be matched for example in relation to different types of head phone, such as “over the ear and noise cancelling”, “over the ear and enclosed”, and insert earpieces, for all of the frequencies and dB levels required.

### Accurate Calibration: Sound Card

In an online hearing test, another factor which can be difficult to take into account in practice is the sound card of the computer at the receiving station 105, 120. To a first order of accuracy, the user can be instructed to set the playback volume at the receiving station 105, 120 to maximum but this can vary between machines. If an absolute measure of hearing is required, it is possible to calibrate the sound card, and indeed the general audio environment of the user, more accurately as described below.

For accurate calibration, the receiving station also has a telephone 275 which is connectable to the server location either by direct dial or over the Internet 125. The server location is provided with the audio tone receiver 270 mentioned above, which might be for example a telephone 310 in combination with a sound level meter 320 or a VAM 335. Where the audio tone receiver 270 uses a telephone 310, this delivers received sound to a sound level meter 320, which in turn delivers sound level data to the calibration sub-routine 300, for instance to a volume data analyser 325. Where the audio tone receiver 270 is an analogue VAM 335 or similar, this digitises received sound which obviates the need for the sound level meter 320, delivering digital information directly to the calibration sub-routine 300. Either the sound level meter 320 or the VAM 335 can be referred to as a volume data generator for the calibration sub-routine 300. In the calibration routine described below, reference is made to the VAM 335 but this is equivalent to the combination of the telephone 310 and the sound level meter 320.

Referring to FIGS. 2 and 3, an accurate calibration protocol 255 is as follows. This protocol 255 is designed to ensure that whichever combination of headphone or soundcard is used, the user's headphones will deliver sounds to the user's ear which match the sound pressure levels required to correspond to known dBHL (decibel hearing level) levels between for example, 20 dBHL and 80 dBHL
1) The user logs on to, or otherwise accesses, the hearing test equipment 200, 205 at the server 100 and selects a calibration sub-routine 300 of the processor 205. The user is asked to enter data concerning their headphone or earpiece equipment 110, 115 such as make, brand and model and to set the soundcard of the receiving computer 105 to a given figure, and to set to “Off” any special features such as bass boost and the like
2) It is advisable at this point that the user listens through the headphones or earpiece equipment 110, 115 to music or other suitable sound to establish that the headphones or earpiece equipment 110, 115 are working.
3) Headphone or earpiece orientation is established by transmitting an audio tone to each ear in turn with user validation after each presentation.
4) The calibration sub-routine 300 instructs the user to enter the number for their telephone 275
5) The calibration sub-routine 300 transmits a calibration audio tone 245, for example at 2 KHz, to the user's computer 105 which will be played through the headphones or earpiece equipment 110, 115
6) The calibration sub-routine 300 establishes a telephone connection to the user's telephone 275 and directs the user to play the sound from their earpiece 110 or headphones 115 into their telephone 275. Instructions can be given here to mitigate variability in headphone placement in relation to the telephone 275
7) The VAM 335 picks up the incoming sound from the telephone line and the volume data analyser 325 analyses the digitised sound output from the VAM 335 and compares it to a previously established calibration value
8) The calibration sub-routine 300 uses the comparison to run the bar display control output 330 to control the sound card calibration meter 235 viewed on the user's computer 105. As described above, this may be displayed as a bar which has two red areas either side of a green one. A needle indicates a position along the bar. The user can effectively move the needle into the green area by changing the setting of the sound card on their computer 105 and re-testing as the sound card is part of a feedback loop via the telephone connection and the VAM 335
9) Upon achieving movement of the needle into the green area, the user presses “Enter”. Because the green area of the sound card calibration meter 235 has been designed to indicate a known sound pressure level received via the VAM 335, the sound level in the earpiece 110 or headphones 115 can now be matched to a known dBHL value
10) The steps above are repeated after adjustment of the user's sound card setting, to confirm correct adjustment Calibration audio tones 245 of different frequencies can be used but it might be noted that, the lower the frequency, the more accurate is the sampled amplitude. Low frequencies, such as 500 Hz to 1 KHz tones, can potentially be interpreted by the VAM 335 as a busy tone in some circumstances. In the calibration regime described above, this is easily avoided by the timing of the interactions.

Calibration values are referred to in step 7 above. In the section entitled “CALIBRATION BY REFERENCE: Headphones and earpieces” above, the development of a library of audio tones 245 in the database 130 matched to different headphones and earpieces is described. The calibration values of step 7 can be established by transmitting one or more of these matched audio tones by telephone to the VAM 335 in the same manner as in steps 6 and 7 described above.

Further in step 7) above, the volume data analyser 325 analyses the digitised sound output from the VAM 335. In more detail, this process is as follows:

7.1) the digital output of the VAM 335 comprises sound files in .wav format (relating to the Waveform Audio File standard) covering one second intervals
7.2) the volume data analyser 325 samples the sound files at ¼, ½ and ¾ of the way through each one second interval
7.3) for each sample, the analyser 325 records the frequency, and the amplitude as a percentage of the maximum possible amplitude
7.4) for each one second sound file, the frequency and amplitude values are averaged. This can be repeated over several sound files for added accuracy
7.5) the analyser 325 checks that the averaged frequency is within a predetermined tolerance which means either that a following hearing test will be useful for its intended purpose or that it might be necessary to establish a frequency profile for the headphone or earpiece equipment 110, 115 so that a frequency offset can be built into subsequent delivery of audio tones 245 for hearing tests to that equipment
7.6) the analyser 325 compares the averaged amplitude values to the previously established calibration value mentioned above in step 7)

The sound files in step 7.2 above might be in for example 8-bit unsigned PCM (pulse code modulation) format.

In steps 8 and 9 above, the calibration sub-routine 300 interacts with the user to adjust a display on the user's computer 105 in setting the sound card volume. This can be avoided by instead downloading a Java applet (or similar locally executable software) to the user's computer 105 which will achieve the same adjustment of the sound card volume without the need for the sound card calibration meter 235 or user inputs. The comparison at step 7 is used to generate volume control data or commands which can be sent to the user's computer 105 where the Java applet (or other local control process) implements sound card volume changes directly, based on the data or commands.

For the purpose of accuracy, the above calibration routine could be carried out for each frequency to be tested.

Where soundcard variances are fairly accurately known, and for example in the case of industrial testing where it is known which headphones are to be used, the above process might be used for validation rather than calibration. It is possible to create reference data comprising a headphone/earpiece profile 265 covering the frequency profile and an expected VAM amplitude for types of user equipment to be encountered. When a user wishes to run a test, the calibration sub-routine 300 will check that the received sound frequency is within tolerance for perhaps one or two calibration audio tones 245 and then compare the received VAM percentage amplitude with the figure stored in the appropriate headphone/earpiece profile 265. It will then either use the Java applet mentioned above to adjust the sound card volume, or instruct the bar display control output 330 to show any amplitude discrepancy on the sound card calibration meter 235 so that the user will increase/decrease their sound card volume by a calculated factor. The sub-routine 300 then repeats the amplitude calibration sound playing/analysis until the desired amplitude is obtained—within a suitable tolerance. Then an actual hearing test can progress.

### Accurate Calibration: Headphone/Ear Phone

Traditional pure tone audiometry is carried out using an annually calibrated audiometer whereby pure tones denoted by dB HL levels are delivered from a known headphone to the individual being tested. However, the actual energy that is presented at the ear does not necessarily match the calibrated dB HL equivalent value due to variances in headphone placement, headband tension, physiological factors relating to the person being tested (head size, meatus and concha shape etc) and the natural “drift” from the original calibration that occurs during the 12 month period leading up to the expiration of the calibration certificate. Further:
1) A human ear's ability to first detect a sound is not uniform across all frequencies. More energy is required below 400 Hz and above 6000 Hz, on average, than for the mid frequencies.
2) Hearing levels are described in dBHL units which are referenced to 0 dB HL which describes the point at which a person first hears a sound at a given frequency. For example 0 dB HL at 250 Hz=approx 18 dBSPL (SPL indicating sound pressure level) whereas 0 dB HL at 2000 Hz is very close to 0 dBSPL.
3) Equal units of sound energy in dBSPL will give rise to differing levels of energy at an ear depending on whether the sound is delivered in a free field (Minimum Audible Field Threshold—MAF) environment or via headphones/ear phones (Minimum Audible Pressure Threshold—MAP).
4) The levels described in (1) arise independently of the type of source of sound but headphones/ear phones are used to measure hearing which utilise MAP levels.

The effect of these variables can contribute to deviances in energy presented at the ear of more than 15 dBSPL which together with the accepted 5 dB HL differential that occurs due to concentration levels or differing ambient noise conditions for example, combine to ensure that pure tone audiometry can deliver results which can be +/−20 dB HL to the intended signal presentation. This problem is particularly prevalent in the high and low frequency measurements.

In light of the above, there is a need for headphones and ear phones to be calibrated to try to ensure an intended dBSPL output from them corresponds to the required dBHL level. Known calibration for this purpose is carried out with the listener absent and specific headphones types are calibrated to a coupler pressure rather than the individual eardrum pressure using a standardised procedure. This enables a Reference Equivalent Threshold Sound Pressure Level (RETSPL) to be calculated—a compensation factor—which is used to calibrate the headphone type to which it refers. But a calibrated audiometer's output may not translate into the intended output at a real ear. Measurement integrity will only be achieved in practice if, in a hearing test, a listener's ear matches the coupler in volume and compliance characteristics and the placement and tension of the headphones is identical to the placement and tension used when calibrating. Thus it is impossible to state with certainty that audiometry will be accurate in relation to a subject, even using a calibrated audiometer. If it is not, there is no way of knowing which frequency is out or by how much.

Referring to FIGS. 5A and 5B, in embodiments of the invention an online test using a microphone 510 mounted in headphones or an insert earphone can provide accurate calibration to mitigate the above problems. The test measures and records the output of the headphone at the person's ear and delivers a true dBSPL value. This true value is used against a reference test tone in real time as outlined below for the audio tone selector 200 to select a correct matrix of audio tone values to enable a pure tone test to be carried out which produces an accurate audiogram in dBHL values at specified frequencies. Although this involves the provision of modified headphones or ear phones, in the case of certain categories such as professionals who carry out hearing screening on a regular basis at a single location, perhaps doctors' surgeries or health surveillance companies who carry out industrial noise testing, it might be particularly suitable to mitigate the problems described above.

Where the headphones are concerned, the microphone 510 is placed within the outer ring 505 of the earward surface of the cup 520. For the insert ear phones, the microphone 510 has a pickup tube 525 extending along the insert tip 530, next to the speaker inlet 515. The microphone 510 is connected to a standard sound input of the person's PC so that the sound level picked up can be dealt with as required.

In practice, the parameters of a test tone to be output by the hearing test system 100 at the headphones or insert ear phones 115, 110 in a calibration exercise will include not only the sound file or audio tone 245 initially selected but also other gain or volume settings. These factors need to be set at known values, for instance as follows:
selected audio tone 245 (therefore frequency and modulation level)
microphone gain control level (equivalent to a Line-In Gain Control on a Mixer console)
loudspeaker volume level, in this case the sound card on the user's PC

The calibration routine can be outlined as follows:
1) A person to be tested places/inserts the headphones 115 or ear phones 110 as for a hearing test and initiates calibration
2) If not already present, the system 100 invites the user to download a Java applet (“executable” or other suitable, downloadable software). Once downloaded, the applet reads the user's current soundcard setting, stores the information on the PC and then sets the soundcard to an appropriate test mode, such as to pre-selected default levels for bass boost etc.
3) The hearing test system 100 will deliver, online, a reference test tone at a given frequency to the sound output 515 of the head/ear phone 115, 110
4) The microphone 510 receives the test tone as it is output at the ear and transmits that sound to the processor 205 to give a measured dBSPL value and, by implication, an actual dBHL value. The processor 205 compares the measured value to a required value.
5) If there is any deviation, the processor 205 uses the Java applet as described above to adjust the PC's soundcard automatically by an amount to match the actual dBSPL output of the headphone or ear phones to the value required to achieve the reference test tone.
6) This exercise is carried out for each of the frequencies to be tested.
7) The processor 205 records and stores each sound card adjustment against the reference test tone in a relevant headphone/earpiece profile 265.
8) Upon completion of the test the processor 205 uses the Java applet to restore the user's soundcard to its original setting.

In an alternative calibration routine, the test is carried out using known headphones which incorporate a sound card 525 working via a USB (Universal Serial Bus) connection 530. When the user plugs the headphones in to a computer and connects to the test, the software does as follows:
1) Detects the headphone soundcard 525 and selects it for use during the test
2) Reads the computer soundcard, stores the settings then mutes it
3) Checks the mic performance on each side (with the cups held together to produce a makeshift “calibration chamber”) by playing a reference test tone from one side which the mic picks up. By running four tests (left headphone/right mic, right headphone left mic, left headphone/left mic, right headphone/right mic) each component could be validated or identified as faulty.
4) The user puts the headphones on and the system plays a reference test tone into one ear which the mic listens to
5) If the sound at the ear doesn't match the reference test tone the system adjusts the gain control on the soundcard 525 until it does match
6) The process is repeated for each frequency to be tested for each ear.
7) At the end of the test the system de selects the headphone soundcard 525 and resets the computer card as default, un-muting it and restoring previous settings.

In calibration, there are four elements to consider:
the level of audio tone 245 selected by the audio tone selector 200 of the hearing test system 100
sound card level at the person's PC
dBSPL values required at the ear
dBHL values that correlate with those dBSPL values

The following describes an example headphone calibration exercise at 1000 Hz with known microphone gain and an initial soundcard level of 75. Assuming the relevant headphone profile 265 indicates that the MAP threshold for the headphones is +7 dBSPL, to present a 75 dBHL tone at an ear the audio tone selector 200 can be expected to need to deliver 82 dBSPL to the ear. An example of a headphone profile 265 at a soundcard level of 75 yields the following extract at 1000 Hz:

TABLE 1

| Required dBSPL | Sounder level | Identity |
|---|---|---|
| 85 | −24/66 | A |
| 84 | /60 | B |
| 83 | /55 | C |
| 82 | /49 | D = 75 dBHL |
| 81 | /44 | E |
| 80 | /38 | A |
| 79 | /34 | B |
| 78 | /31 | C |
| 77 | /28 | D |
| 76 | /24 | E |
| 75 | /21 | A |

This profile shows a reference test tone for delivering 75 dBHL at −24/49. Thus to achieve an expected 75 dBHL, the Java applet sets the user's PC soundcard to 75 and delivers a reference test tone at −24/49. However, in a calibration exercise the measured dBSPL equivalent figure that is returned via the microphone 510 is not 82 but perhaps 78. Now the system 200, 205 uses the applet to adjust the soundcard until the measured dBSPL is 82, stores the soundcard level and the identity of the reference test tone at −24/49 and then moves on to the next frequency. If the outputs can't be matched, the next best value to +/−3 dBSPL is selected with the appropriate identity stored. If it isn't possible to get a match the test is halted.

The identities A to E of the audio tones identify a range of audio tones 245 with respect to each calibrated reference test tone in a headphone profile 265 which will produce a set of 5 dB reductions in dBSPL. These identities are stored as linked values with respect to each profile 265. Due to the logarithmic nature of the scale it is known that each 5 dBSPL reduction will equal a 5 dBHL reduction down to 20 dBHL. Once a calibrated reference test tone is known, the system 100 can present a series of hearing test tones, each 5 dBHL lower than the last, by simply stepping down a series of the audio tones 245 having the same linked identity value, for instance “A”.

There are two ways the measured sound card level adjustments obtained during calibration can be used in subsequent hearing tests. Either the user's sound card output can be adjusted by the measured amount against each frequency in subsequent hearing tests using the same user equipment or it could be used to select a different audio tone 245 having a different playback volume level to achieve the required dBSPL.

### Hearing Test Processes

A hearing test process is now described below as an example of the equipment in use. The processing of hearing test results is not described here in detail as such processes are known. Embodiments of the invention are more concerned with improving the accuracy of the results and with producing an absolute measure of hearing in spite of potentially major differences between one audio environment and the next. For example, the representation of hearing test results, as recorded on an audiogram using a dBHL (hearing level) scale which is specific to human hearing, produces a simple graph with a vertical intensity scale and a horizontal frequency scale. It is known that adults with hearing thresholds no greater than 20 dBHL are considered to have normal hearing. Two or more frequencies with hearing thresholds greater than 20 dBHL are therefore considered to represent a measurable hearing loss in an adult human.

In the hearing test process, the user logs on to or otherwise accesses the hearing test processor 205 over the Internet 125. The processor 205 responds by acquiring calibration data regarding the user's audio environment, particularly the type of headphones to be used. This might be done as described above, and/or by requesting the information from the user by means of a form or drop-down menu for example, or perhaps by interrogating the computer 105, 120 itself.

The processor 205 instructs the user to increase or decrease the volume setting of the sound card (sound producing device) of their computer 105, 120 either to its calibrated level or to its maximum level, and to set to “Off” any special features such as bass boost and the like. It is advisable at this point that the user listens through the headphones 115 to music or other suitable sound to establish that the headphones 115 are working.

Headphone orientation is established by transmitting an audio tone to each ear in turn with user validation after each presentation. The user is then asked which is their “better” ear and the test commences with the better ear or defaults to the right ear if hearing is equal in each ear.

Tests are then conducted in “phases” for each ear with required frequencies eg 1 Khz, 2 KHz, 4 KHz and 500 Hz—in this instance, a total of 8 phases. In practice, four frequencies is considered the minimum, six or more is not uncommon. The processor 205 communicates with the user by standard means such as transmitting message content, the user responding by entering text or other keystrokes via their keyboard or pointing device or by other means such as via the VAM 335.

The audio tone selector 200 in practice, as described above, is a software process run by the processor 205 each time it is necessary to transmit an audio tone 245 from the database files 215 via the transmitter receiver 240 and over the Internet. The tone selected depends on the progress of a hearing test and particularly on the responses of the user in order to deliver the required frequency and output level. Indeed, the processor 205 itself will be embodied as a co-ordinating software process that calls on sub-processes such as the audio tone selector 200 during use of the hearing test equipment.

Practitioners of the art will be conversant with most of the following procedure which replicates traditional audiometric testing in that the user presses a key every time they hear a presented sound.

The user is instructed to press a key every time a presented tone is perceived with the intention being to establish the quietest tone for a given frequency the user can perceive.

A tone which would be comfortably heard by a person with normal hearing at 1 Khz (eg) 60 dBHL is presented to the user's single pre-selected ear. If the user responds to that tone the test can commence. If the user does not respond to the tone the intensity is increased (eg) 80 dBhl. If the user responds to that tone the test commences at that level. If the user still fails to respond, the system records the value as not heard and moves on with the test at the next frequency.

Assuming the user responds to a presented tone. The process will institute a random tone presentation according to prevailing BSA (British Society of Audiometry) recommended procedures for air conduction, pure tone audiometry. This measures the threshold of sound perception at the presented frequency and this is the value that the process records.

The process will then repeat the above procedure at all of the other frequencies, recording each threshold in turn.

On completion of testing the user's first ear, the process repeats the procedure on the user's other ear.

It is known to practitioners of the art that some users have difficulty is assimilating the test procedure instructions. In these and other instances the process of the current invention permits the user or the process to define by selection other methods of achieving the requisite results. One such method is described below as an example.

After the introductory procedures as described above, a tone which would be comfortably heard by a person with normal hearing at 1 Khz (eg) 60 dBhl is presented to the user's single pre-selected ear. If the user responds to that tone the test can commence. If the user does not respond to the tone the intensity is increased (eg) 80 dBhl. If the user responds to that tone the test commences at that level. If the user still fails to respond, the system records the value as not heard and moves on with the test at the next frequency.

Assuming the user affirms hearing the tone presented, they are then instructed to count the number of tones which are presented at the given frequency. The tones are presented sometimes duplicated with between 1 and 3 seconds delay between presentations, each presentation decreasing by 5 dBHL. On completion of the presentations, the user is requested to select a number which equates to the number they have counted. This number is stored within the process and converted into the threshold level achieved by the user.

On completion of the test in whichever form, the user is requested to fill in their name and email address so that the results can be sent to them. and/or stored.

An alternative test method is to ask the user to press a button on the screen each time a tone is presented.

Key features of embodiments of the invention are:
a) The user can access and self test at any time and date to suit them.
b) The test is simple to use.
c) The reading obtained may be used in the same way as a conventional test.
d) This invention can be used for industrial noise testing.
e) No special equipment is needed (except where high accuracy is required and therefore calibrated headphones/insert ear phones 115, 100 are to be used)
f) No special skills are required.
g) The results are presented in an easy to understand format
h) The calibration/validation routines can be rendered very accurate

It is believed that embodiments of the invention can produce an online test which is accurate enough to be used in the industrial noise testing arena. For industrial testing however, it may be necessary to use specific headphones thereby removing at a stroke a major variable.

Embodiments of the invention support any means of producing and recording (or not) in any form and for any duration of time, sounds which are responded to at any time and by any method by a person or persons which may be deemed a hearing test, check evaluation or any other term of application or presentation by or from a computer or external source where the operator or producer is effecting or presenting any form of automatic, manual, recorded or live presentation to which a user or users may respond over any distance via a computer or external source where the user is not attached by the traditional means to a device for testing, checking, evaluating etc.

The end of the test permits the user access to the results which may be shown on screen, emailed or sent by other means to the recipient or stored for future reference.

1. Hearing test apparatus for transmitting audio tones over a network for playing as audible sound at a receiving station and for receiving inputs over the network in relation to the audible sound, the apparatus comprising:
i) an audio tone store for storing recorded audio tones,
ii) a transmitter for transmitting selected tones via the network to the receiving station,
iii) a receiver for receiving inputs from the receiving station via the network in relation to transmitted audio tones, and
iv) a processor for processing and responding to received inputs so as to carry out a hearing test and generate hearing test results,
wherein the audio tone store is structured to store each audio tone as a set of two or more audio tone components having the same frequency, each component having a different modulation level, and the apparatus further comprises:
iii) an audio tone selector for selecting in accordance with received inputs, for each transmitted audio tone, one or more audio tone components from one of said sets for transmission via the network.

2. Hearing test apparatus according to claim 1 wherein the audio tone store is structured to store each set of audio tone components as a set of at least two sound files at the same frequency, each sound file of a set having been recorded at a different modulation level.

3. Hearing test apparatus according to claim 1, further comprising an input for receiving volume control data from the receiving station, in use of the apparatus, the audio tone selector being configured to process received volume control data for use in selecting the audio tone components and/or output levels of each component, to be transmitted as an audio tone via the network.

4. Hearing test apparatus according to claim 1, further comprising an input for receiving calibration data from the receiving station, in use of the apparatus, the audio tone selector being configured to process received calibration data for use in selecting the audio tone components and/or output levels of each component, to be transmitted as an audio tone via the network.

5. Hearing test apparatus according to claim 1, further comprising an input for receiving notification from the receiving station, during transmission of a current audio tone to the receiving station, and a processor for responding to receipt of a notification so as to store data for use in assembling a hearing test result.

6. Hearing test apparatus according to claim 1, further comprising a reference data store, accessible to or stored at the transmission apparatus, for use by the audio tone selector in selecting the audio tone components to be transmitted via the network.

7. Hearing test apparatus according to claim 1, wherein each set of audio tone components having the same frequency comprises at least one component recorded with modulation of at least 90% and further components recorded with modulation in each of the ranges 1.0-10%, 0.1-1.0% and less than 0.1%.

8. Hearing test apparatus according to claim 7, wherein each set of audio tone components having the same frequency comprises at least four components.

9. Hearing test apparatus for transmitting audio tones over a network for playing as audible sound at a receiving station and for receiving inputs over the network in relation to the audible sound, further comprising calibration apparatus for use in calibrating the volume of audible output of the receiving station in response to one or more calibration audio tones transmitted by the transmitter, the calibration apparatus comprising:
a) an audio tone input for receiving a copy of the audible output from the receiving station; and
b) a volume data generator for generating volume data from the received copy,
the calibration apparatus being arranged to provide, in use, a feedback loop in which a copy of the audible output of the receiving station, in response to one or more calibration audio tones transmitted by the transmitter, is returned to the audio tone transmission apparatus, and used to generate volume data for use in turn at the receiving station in adjusting one or more sound output levels of the receiving station so as to calibrate the volume of the audible output of the receiving station.

10. Hearing test apparatus according to claim 9, further comprising a volume display output for making volume display data accessible to the receiving station in accordance with the volume data, for use at the receiving station by means of user inputs in said adjustment of one or more sound output levels of the receiving station.

11. Hearing test apparatus according to claim 1, further comprising calibration apparatus for use in calibrating the volume of audible output of the receiving station in response to one or more calibration audio tones transmitted by the transmitter, the calibration apparatus comprising:
a) an audio tone input for receiving a copy of the audible output from the receiving station;
b) a volume data generator for generating volume data from the received copy, and
c) a volume control output for transmitting volume control data or commands to the receiving station in accordance with the volume data,
the calibration apparatus being arranged to provide, in use, a feedback loop in which the copy of the audible output of the receiving station, received in response to one or more calibration audio tones transmitted by the transmitter, is used at the audio tone transmission apparatus to generate volume data which can be transmitted in turn, as volume control data or commands, to the receiving station to adjust one or more sound output levels of the receiving station so as to calibrate the volume of the audible output of the receiving station.

12. Hearing test apparatus according to claim 9, wherein the audio tone input comprises a telephone receiver.

13. Hearing test apparatus according to claim 9, wherein the audio tone input comprises a voice activated modem.

14. Hearing test apparatus according to claim 9, wherein the volume data generator is provided with calibration values for use in generating the volume data from the received copy of the audible output from the receiving station.

15. Hearing test apparatus comprising:
i) a transmitter for transmitting selected audio tones having different respective volumes via a network to a receiving station,
ii) audio delivery equipment for use at the receiving station in delivering audio tones to the user's ear,
iii) a receiver for receiving inputs from the receiving station via the network in relation to transmitted audio tones, and
v) a processor for processing and responding to received inputs so as to carry out a hearing test,
the hearing test apparatus further comprising calibration apparatus for use in calibrating the volume of delivered audible output to a user's ear in response to audio tones transmitted by the transmitter, wherein the audio delivery equipment comprises an audio pickup for picking up transmitted audio tones at delivery to the user's ear, for use in providing feedback to the processor enabling volume calibration of the audio tones in relation to the audio delivery equipment in use.

16. Hearing test apparatus according to claim 15, further comprising a volume adjustment controller for adjusting one or more sound output levels of the audio delivery equipment for use in calibrating the volume of delivered audible output to a user's ear in response to audio tones transmitted by the transmitter.

17. Hearing test apparatus according to claim 16 wherein the audio delivery equipment comprises headphones.

18. Hearing test apparatus according to claim 16 wherein the audio delivery equipment comprises an insert ear phone.

19. Hearing test apparatus according to claim 16, wherein the audio pickup comprises a microphone mounted in the audio delivery equipment for picking up audio tones at the point of delivery to the user's ear for use in providing a volume indication with respect to delivered audio tones.

20. Hearing test apparatus according to claim 16, wherein the audio delivery equipment comprises a sound card and the volume adjustment controller is configured to mute a sound card of the receiving station and to adjust one or more sound output levels of the audio delivery equipment in calibrating the volume of delivered audible output to a user's ear.
